(19) United States
(12) Patent Application Publication
(10) Pub. No.: US 2022/0327951 A1
(43) Pub. Date: Oct. 13, 2022

(54) Hearing Screening Simulator Application

(71) Applicant: University of South Carolina, Columbia, SC (US)

(72) Inventors: John H. Gerdes, Cayce, SC (US); Dakota Sharp, Columbia, SC (US)

(73) Assignee: University of South Carolina, Columbia, SC (US)

(21) Appl. No.: 17/717,342

(22) Filed: Apr. 11, 2022

Related U.S. Application Data
(60) Provisional application No. 63/174,158, filed on Apr. 13, 2021.

(51) Int. Cl. G09B 9/00; G06T 13/80; A61B 5/12; A61B 5/00

(52) U.S. Cl. CPC G09B 9/00; G06T 13/80; A61B 5/123; A61B 5/7475

(57) ABSTRACT

Described herein is a software-based simulator to provide speech and hearing students the ability to perform hearing screening training using a realistic interface similar to devices used to perform these tests, which provides results for Otoscopy, Tympanometry, and Audiometry testing, and assesses the students responses for correctness.

### TECHNICAL FIELD

The subject matter disclosed herein is generally directed to a software-based simulator to provide speech and hearing students the ability to perform hearing screening training using a realistic interface similar to devices used to perform these tests, which provides results for Otoscopy, Tympanometry, and Audiometry testing, and assesses the students responses for correctness.

### BACKGROUND

There are over three hundred (300) institutions offering degree programs in audiology, speech-language pathology, and speech, language, and hearing science. The 2014 CSD Education Survey indicates that over 38,000 students were enrolled in undergraduate Communication Sciences & Disorders majors, with over 16,000 enrolled Master's Speech-Language Pathology students, and over 2500 Audiology clinical doctorate students. Both of these fields are predicted to experience significant growth over the next 10 years.

Speech-language pathologists conduct hearing screening tests to attempt to identify hearing problems early. If a potential problem is identified, the subject is sent for a full hearing evaluation. However, with respect to the current state of the art, there is no existing system to provide training except using physical devices. This is expensive and does not address remote learners. Further, systems that must be installed on a computer are also inconvenient and possibly limiting in their scope and reach to those being trained.

Accordingly, it is an object of the present disclosure to provide a web-based application, which would significantly improve the usability and reach of the system to prospective trainees.

Citation or identification of any document in this application is not an admission that such a document is available as prior art to the present disclosure.

### SUMMARY

The above objectives are accomplished according to the present disclosure by providing in one instance, an audiology simulator. The audiology simulator may include an otoscopy component to provide a visual representation of at least one ear, a tympanometry measuring component to simulate measurement of eardrum movement in the at least one ear; and an audiometer component to simulate measuring hearing in the at least one ear; and wherein the otoscopy component, the tympanometry measuring component, and the audiometer component replicate administering and interpreting a hearing test given to a subject. Further, the audiology simulator may provide at least one result of an operator's performance using the audiology simulator. Still, the audiology simulator may enable cross-sectional data analysis across multiple users to compare different users' results using the audiology simulator. Moreover, the audiology simulator may include a graphical interface. Yet again, the graphical interface may provide at least one display output that includes at least one otoscope image, at least one tympanogram, and at least one audiometer chart. Again yet, the audiology simulator may be web-based. Further again, the audiology simulator may include a subject information section providing information about the subject. Still again further, the audiology simulator may provide sensory input to a user via interactive objects to confirm an action performed by the audiology simulator. Still moreover, the audiology simulator may include an avatar representing the subject being tested by the audiology simulator wherein the avatar provides visual cues to a user to show the avatar receiving sensory input from the audiology simulator.

In a further instance, the current disclosure may provide an auditory training simulation method. The method may include providing an audiology simulator that may include an otoscopy component to provide a visual representation of at least one ear; a tympanometry measuring component to measure eardrum movement in the at least one ear; and an audiometer component to measure hearing in the at least one ear; and replicating administering and interpreting a hearing test given to a subject via the otoscopy component, the tympanometry measuring component, and the audiometer component. Still further, the method may include providing via the audiology simulator at least one result of an operator's performance using the audiology simulator. Yet again, the method may, enable, via the audiology simulator, cross-sectional data analysis across multiple users to compare different users' results using the audiology simulator. Further yet, the method may include providing a graphical interface to interact with a user. Moreover, the method may provide via the graphical interface at least one display output that includes at least one otoscope image, at least one tympanogram, and at least one audiometer chart. Yet further, the method may provide the audiology simulator as a web-based platform. Still further, the method may provide a subject information section that provides information about the subject. Even further, the method may provide sensory input, via the audiology simulator, to a user via interactive objects to confirm an action performed by the audiology simulator. Still even further, the method may, via the audiology simulator, presented an avatar representing the subject being tested by the audiology simulator wherein the avatar provides visual cues to a user to show the avatar receiving sensory input from the audiology simulator.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows one embodiment of a user interface of the current disclosure.

FIG. 2A shows instructions on how to use the user interface of the current disclosure.

FIG. 2B shows a list of potential test subjects that may be selected for use with the interface.

FIG. 2C shows a sample bio that may be provided to the trainee describing the test subject selected.

FIG. 2D shows one example of actual ear/auditory canal images that may be presented to a trainee using an interface of the current disclosure.

FIG. 2E shows an example of tympanometry results presented to the trainee during use of the interface.

FIG. 3 shows a representation of a testing subject that may provide visual cues to trainees using the interface of the current disclosure.

FIG. 4 shows a representation of a testing subject responding to audio stimuli supplied via the user interface.

FIG. 5 shows audiometry simulation results generated by a user interface of the current disclosure.

FIG. 6 shows a results section of one embodiment of the current disclosure.

FIG. 7A shows one portion of a report generated from testing a test subject via the current disclosure.

FIG. 7B shows one example of trainee results that may be displayed by comparing the trainee's diagnosis to the actual conditions of the test subject.

FIG. 8 shows at: (a) an otoscope image of a right ear; (b) a tympanogram trace for a right ear; and (c) an audiogram for a right and left ear.

FIG. 9 shows a simulation summary for a hypothetical patient.

FIG. 10 shows Table 1—Technique Assessment by Certified Technicians in Live Testing Using Commercial Audiometry Equipment.

FIG. 11 shows Table 2—Multi-Factor Assessment of Student Confidence Change. Pre-Test and Post-Test Values Represent the Average from Both Control and Test Groups.

FIG. 12 shows Table 3—Individual Survey Item Analysis Grouped by Otoscopy, Tympanogram, and Hearing Screening Questions.

FIG. 13 shows one example of an avatar of the current disclosure.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

Early identification of hearing impairment and ear disorders is important, which is why hearing screening is routinely done on newborns, with regular screening recommended on children through the age of 18. Screening is also completed with adults to assess and treat hearing problems. Procedural training is needed for new Speech-Language Pathologists and nursing students as well as continuing education for those trained to perform this procedure. An audiology simulator was developed to provide an alternative to traditional face-to-face lab instruction. Using a design science approach, the development of the simulation prototype is discussed. Contributions include a useful framework for developing such a simulation of an existing process, a description of a unique artifact that supports an individualized, self-paced learning environment using context-sensitive feedback and performance assessment, and an extensible approach to supporting virtual subjects in audiological training.

The current disclosure provides a Software Simulation of a Hearing Screening educational trainer, requiring the “technician” learner to assess a series of Otoscope images, Tympanometry traces, and subject Audiometer responses, each associated with a virtual subject. The technician performs the tests and adds their assessment, diagnosis, and notes related to each test. Once completed, the system checks the technician's assessments against the correct answers for that subject and provides feedback to the technician learner. The current disclosure provides the ability to do randomized tests, and has the ability to replicate tests for inter-reliability testing. This application is primarily targeted to students in the field of communication disorders (speech-language pathologists and audiologists), but could be used in training nurses, teachers, and others involved in conducting hearing screenings.

The current disclosure automatically assesses learners and automatically provides feedback. Learners do not need supervision, which is significant due to the shortage of faculty, and the application can run on any windows computer, including a computer lab, making the training more accessible to trainees.

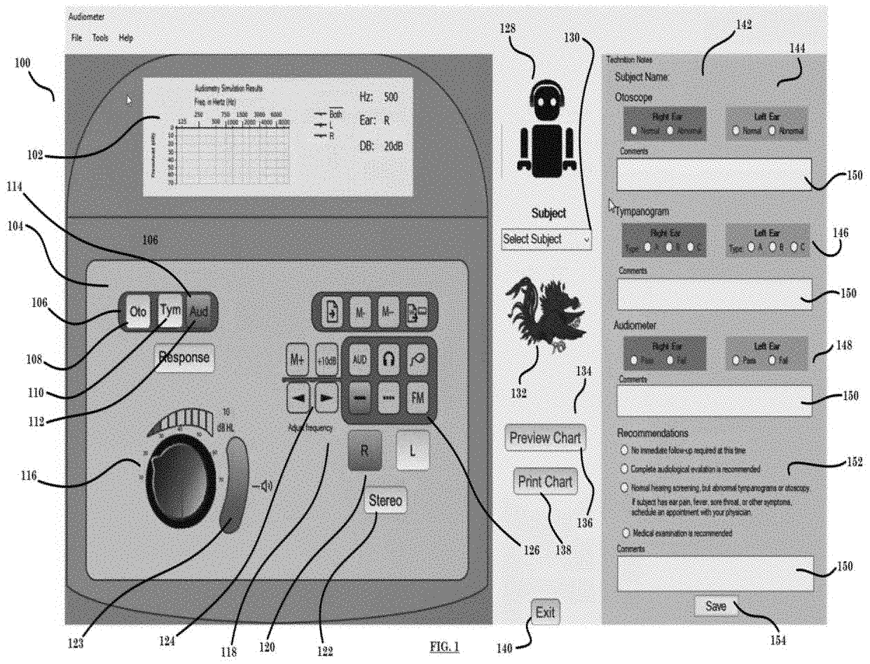

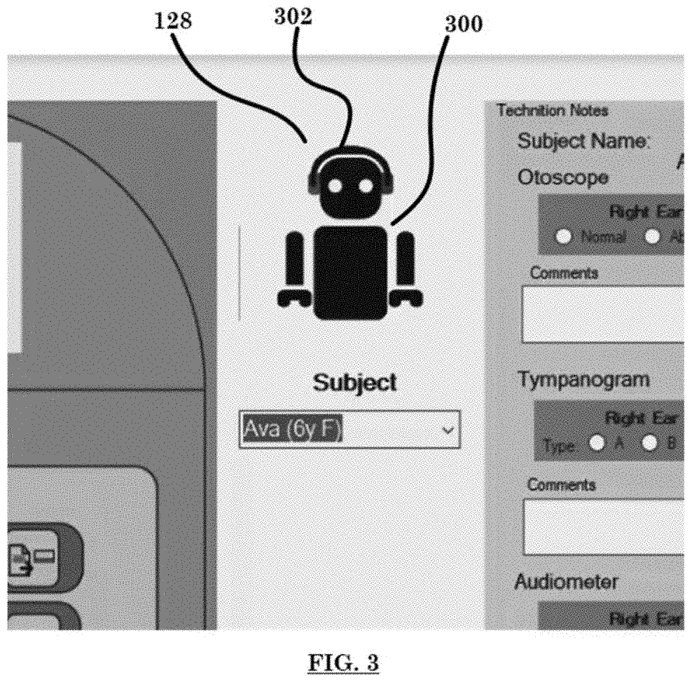

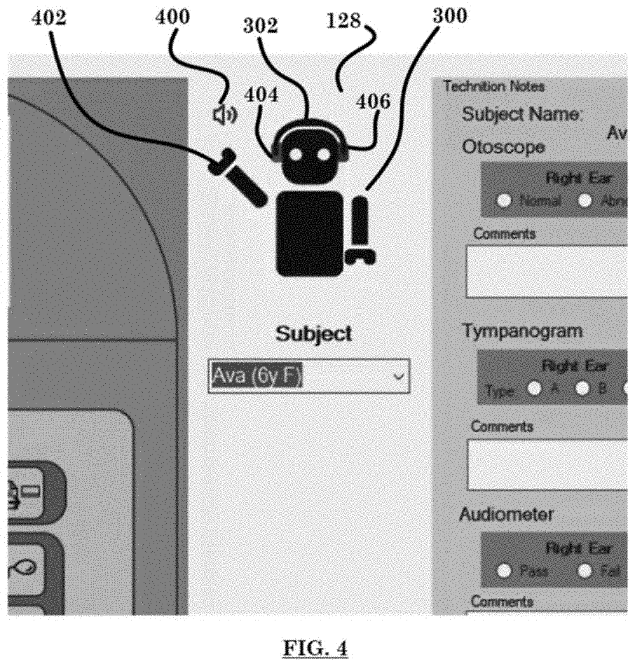

FIG. 1 shows one embodiment of a user interface 100 for the current disclosure. User interface 100 may include graphical display 102 to display testing results testing controls 104. Testing controls 104 may include test selection feature 106 that allows the trainee to select the test(s) being applied such as Otoscope 108, Tympanogram 110 and/or Audiometer 112. Selected features on the user interface may show a visual cue 114, such as changing color, flashing, etc., to indicate which test has been selected and is being performed, which ear is being tested, etc. User interface 100 may also include analog representation 116 of dials, knobs, power meters, etc., that appear on the actual equipment to make interface 100 appear like the actual piece of equipment, which may receive two dimensional input such as twisting, pushing a knob, flipping a switch, etc., that the trainee would encounter during use of the actual equipment for testing a live patient. User interface 100 may also include system control representations 118 that may include ear selection 120 for choosing to test either the right or left ear or stereo testing 122 for both ears simultaneously, present tone control 123 for supplying a selected frequency to either one or both ears of testing subject representation 128, frequency adjustment 124, as well as various user controls 126. User interface may also include testing subject representation 128, which may appear as a stylized body or portion of a body, that may interact with the trainee similarly to a live patient, see FIGS. 3 and 4 infra. User interface 100 may also include subject selection 130 to pick from a variety of test subjects that represent individuals with normal, abnormal, damaged, changing hearing in order to represent the types of patients the trainee will meet during live testing of actual living subjects. User interface 100 may also include branding 132, herein the University of South Carolina Gamecocks emblem, to allow companies or organizations using interface 100 to personalize same to their particular business, industry, etc. Interface 100 may also include chart options 134 such as previewing 136 test subject charts and/or printing charts 138 as well as an exit selection 140 to allow the trainee to exit the interface. Interface 100 may also include technician notes 142 that allow a trainee to diagnosis either ear under various testing scenarios such as otoscope testing 144, tympanogram testing 146, audiometer testing 148, etc. The trainee may comment 150 on the results of each test as well as provide treatment recommendations 152 with respect to each test subject tested. User interface 100 may also allow the trainee to save 154 the test results for further review and/or comparison to previous or later conducted testing.

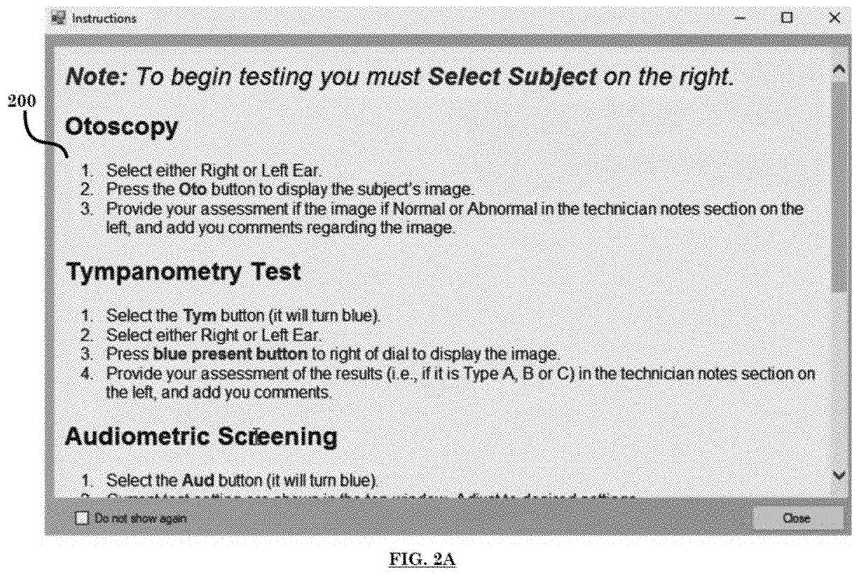

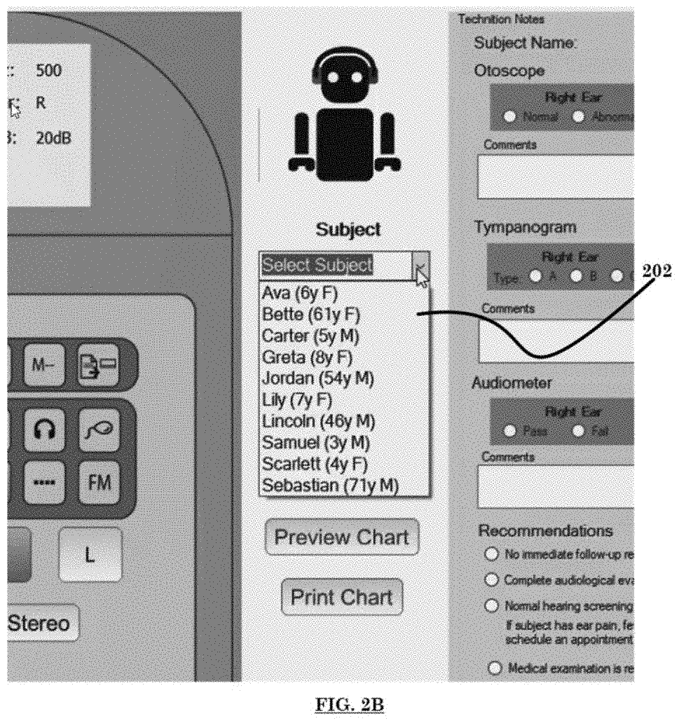

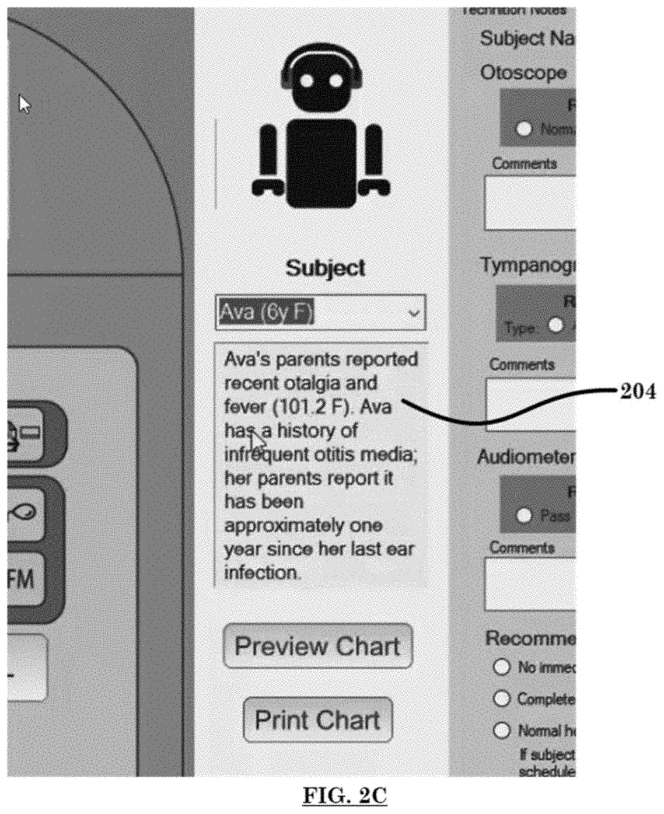

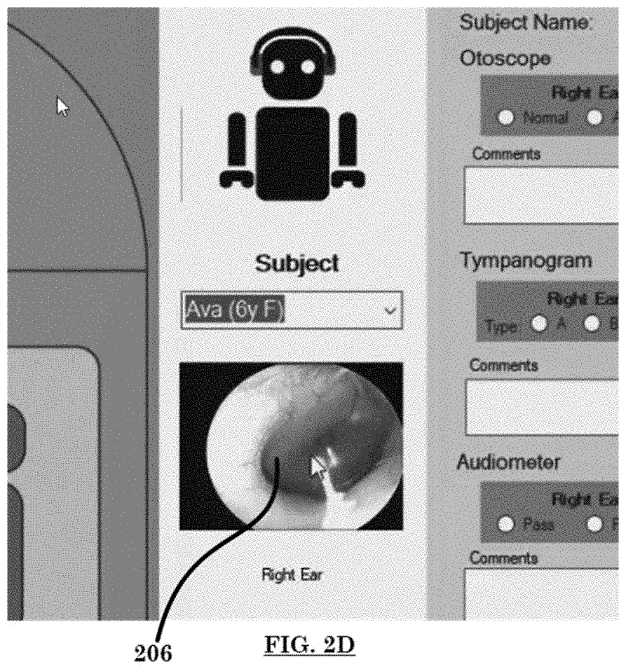

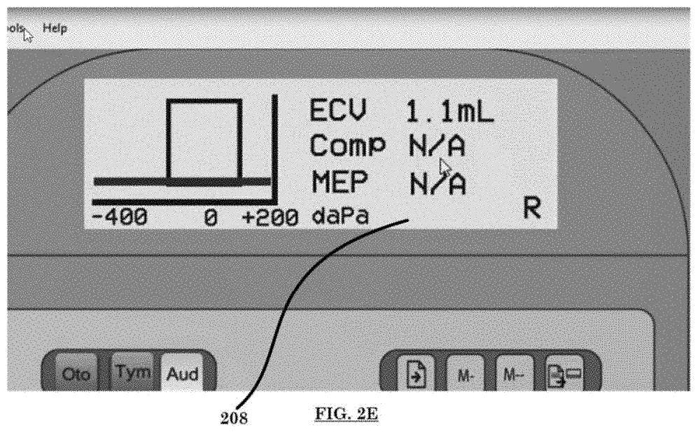

In one embodiment, a trainee could log onto the hearing screening simulator system. The trainee would then be presented with instructions 200, see FIG. 2A, explaining how to operate the simulator. The trainee would then be provided, see FIG. 2B, a list of hypothetical test subjects 202 for use with the system. The system could also offer a test subject bio 204, see FIG. 2C, of the test subject providing medical details, past hearing analysis results, current prognosis, diagnosis suggestions, etc., for the test subject the trainee selected at FIG. 2B. The trainee could then select from a group of tests to run on the hypothetical patient. This could include otoscope testing, which provides actual patient images 206, see FIG. 2D, of the inner ear, for either ear, of the hypothetical test subject. This allows the trainee to see an actual human ear displayed on interface 100 that may or may not have audio and/or physical issues to allow the trainee, via interface 100, to experience what they would see during actual patient testing and examination.

The testing can also involve a Tympanogram step, where a test is ran and measures the function and movement of the eardrum and middle ear. The results of tympanometry could be represented via a visual illustration 208, see FIG. 2E, called a tympanogram, which is displayed to the trainee on user interface 100. One important aspect of user interface 100 is that it not only can show actual ear conditions to the trainee but the results provided by user interface 100 to the trainee would mimic or be identical to those shown on the actual testing device used with a live patent. This promotes familiarity and experience with the testing device without actually having to use a single or multiple devices for all the various tests one might conduct while evaluating a patient's hearing and/or ear condition.

The system could also provide audiometer testing. This would test the ability of the hypothetical test subject to “hear” different frequencies. User interface 100 could allow increasing and decreasing the level of decibels presented to the test subject via interaction with analog representation 116 and/or frequency adjustment 124, such as by clicking on same, sliding a mouse over same, selecting a particular decibel level by clicking on same, etc. In one aspect, the testing subject representation 128 may be a stylized upper torso 300 shown on the system display, see FIG. 3. The stylized upper torso would represent the test subject being in a testing facility with headphones 302 worn to provide the sounds generated during testing. Referring to FIG. 4, user interface 100 could confirm via visual and audio signifiers to the trainee that the sound was being presented to the test subject. This could include the trainee hearing a tone when present tone control 123 was activated and/or employing a visual indication 400 to the trainee that sound was being introduced to the test subject via interface 100. A visual representation of the hypothetical test subject, testing subject representation 128, could be provided to the trainee as well. The appearance of testing subject representation 128 could change to indicate the test subject was “hearing” the sound provide via visual subject indicator 402, herein shown by the test subject raising its “hand” on the display to indicate it has heard the tone being supplied via the system, see FIGS. 3 and 4. Moreover, interface 100 may also include right ear visual cue 404 and left ear visual cue 406 to show which ear is receiving sound via having the visual cue change appearance as sound is applied and for this change to cease when the sound is stopped, such as via a flashing or solid light shown on either or both ears.

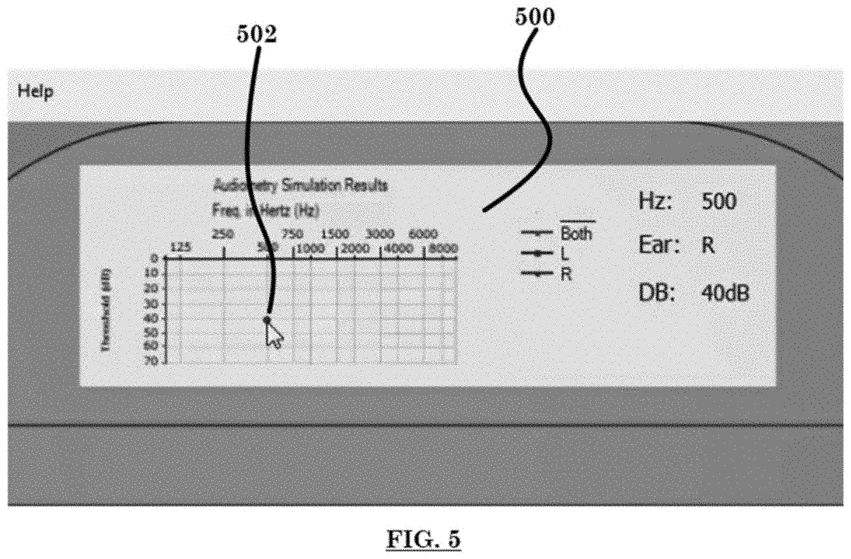

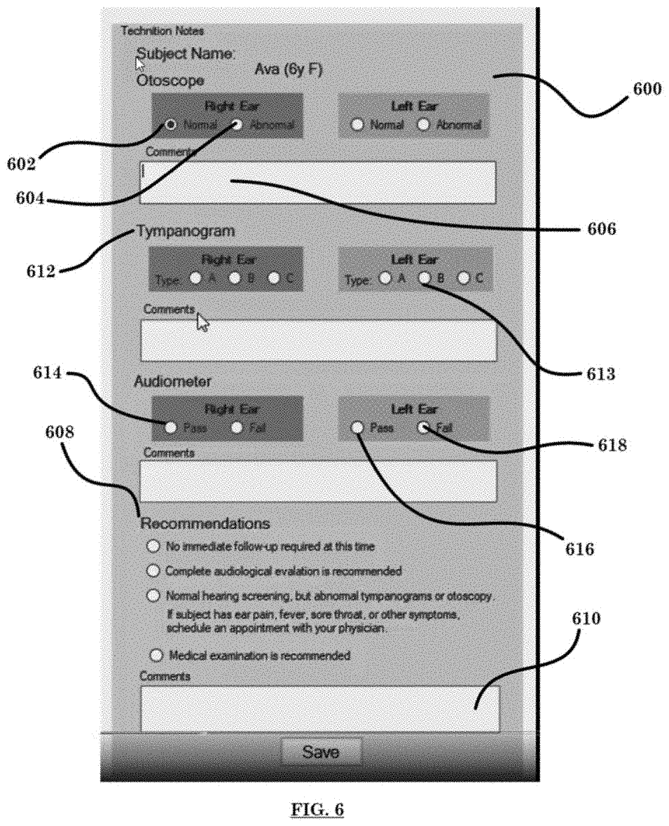

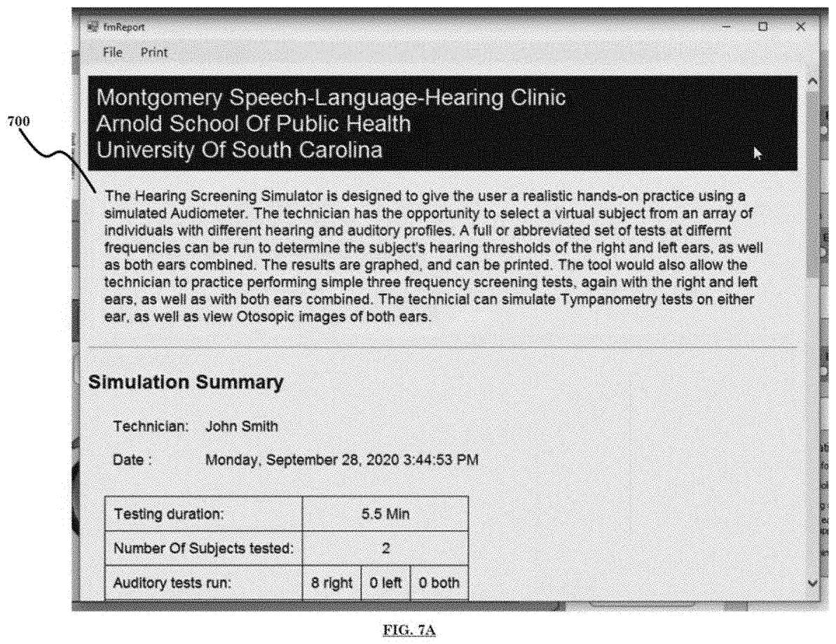

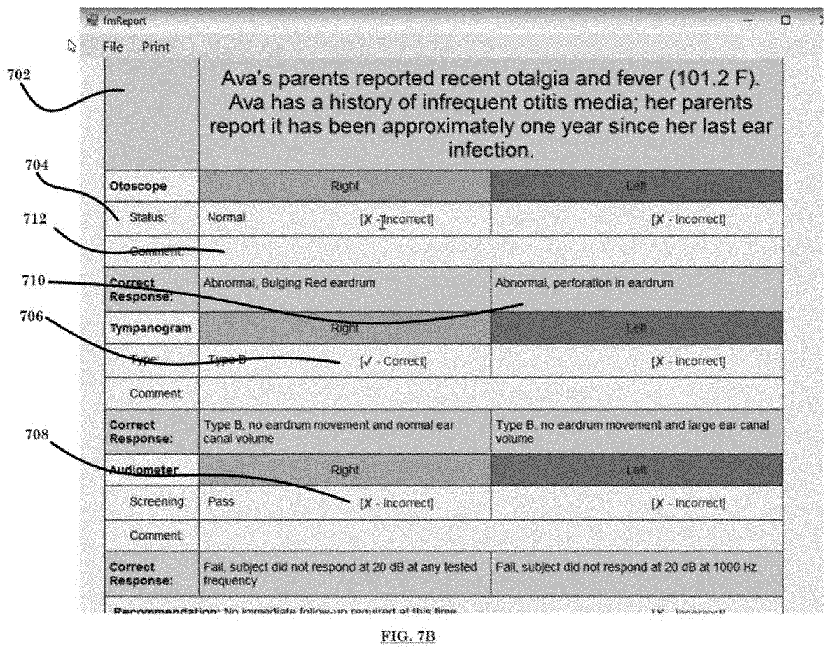

When the sound is “heard” by the test subject, the trainee can then mark 502 this result on the system via interaction with audiometry simulation results 500, see FIG. 5. Once testing is complete, the trainee may indicate the results of each section, see FIG. 6, by indicating in results section 600 of the system whether each ear had normal 602 or abnormal hearing 604 for each type of test, such as selecting tympanogram result 612 and tympanogram result determinations 613 and audiometer results 614 indicating whether the test subject passes 616 or failed 618 for each ear with respect to the audiometer testing. The trainee can perform the abnormal/normal designation as well as insert comments 606 for each test employed via the system. The trainee may also provide treatment recommendations 608 based on the results of the test subjects reactions to each test as well as provide treatment comments 610 about the hypothetical test subject's results. Once results section 600 is finalized, the system can generate report 700, see FIG. 7A, which may be a PDF or other form of electronic document which can be edited, printed, etc., as known to those of skill in the art. FIG. 7B shows a testing results portion 702 of report 700 showing whether the trainee was accurate in assessing the test subjects via providing visual feedback 704 whether the trainee correctly 706 or incorrectly 708 analyzed the responses from the test subject. The system may also provide correct diagnosis information 710 that may be compared to trainee diagnosis responses 712 to show whether the trainee's assessments were accurate or need correction. Visual Basic (VB) may be used to form a visual interface as described and shown herein. In the discussed embodiment, three tests are shown but the current disclosure is not so limited and should not be considered restrained to just these three types of testing as more and varied testing is considered encompassed within this disclosure.

The current disclosure provides an audiology simulator developed to provide an alternative to a traditional face-to-face lab instruction training for Language Speech Pathologists learning how to perform hearing screening assessments. This tool would also be useful for nurses, teachers and audiologists to train on performing this procedure.

The Hearing Screening test is a fast, inexpensive, and easy to perform pass/refer screening procedure used to identify those who require further audiological evaluation. Those who do not pass a hearing screening (the term fail is not used) is typically referred to a licensed audiologist who would perform more detailed hearing testing.

A hearing screening test can involve three procedures—pure-tone hearing screening which identifies hearing impairment, otoscopy which is a visual inspection of the outer ear, and tympanometry which measures the movement of the eardrum in response to a change in pressure. Different protocols are used depending on patient's age. See, American Speech-Language-Hearing Association, “Guidelines for audiologic screening”, 1997. For school-age children, hearing screening helps identify those most likely to have a hearing impairment that may interfere with their education, health, development, or communication. Hearing impairment is defined as the inability to hear a pure tone at the 20 dB HL level. See, Id. Hearing screening is also completed with adults. Hearing impairment (i.e., loss or abnormality of psychological or physiological function) and/or hearing disability (i.e., restriction or lack of ability to perform an activity, resulting from an impairment) are prevalent chronic conditions among adults of all ages with hearing impairment increasing as a function of age.

Guidelines also exist for screening for outer and middle ear disorders. It is a pass/refer procedure that involves a visual inspection via an otoscope or video otoscope, and acoustic immittance testing via a tympanogram. An otoscopy exam is a clinical procedure used to examine structures of the outer ear, particularly the external auditory canal and tympanic membrane (eardrum). A tympanometry test measures how the eardrum moves and helps diagnose disorders that can lead to hearing loss.

Medical institutions are grappling with how decrease training costs with an increased focus on digital education, including online and distance learning programs, and simulations. Simulation is increasingly being used in medical training. It has been found to improve nursing knowledge and skills, self-confidence, communication skills, empathy, critical thinking abilities, leadership, and situation management among nursing students. See, R. P. Cant, and S. J. Cooper, “Simulation-based learning in nurse education: systematic review. Journal of Advanced Nursing, vol. 66, pp. 3-15, 2010, v. Guise, M. Chambers, and M. Välimäki, “What can virtual patient simulation offer mental health nursing education?”, Journal of psychiatric and mental health nursing, vol. 19, no. 5, pp. 410-418, 2012, C. La Cerra, A. Dante, V. Caponnetto, I. Franconi, E. Gaxhja, C. Petrucci, et al., “Effects of high-fidelity simulation based on life-threatening clinical condition scenarios on learning outcomes of undergraduate and postgraduate nursing students: a systematic review and meta-analysis”, BMJ Open, vol. 9, No. 2, e025306, 2019, and D. K. Brown, “Simulation before clinical practice: the educational advantages”, Audiology Today, vol. 29, no. 5, pp. 16-24, 2017, D. Bakhos, J. Galvin, J. M. Aoustin, M. Robier, S. Kerneis, G. Bechet, . . . & Aussedat, C. (2020). “Training outcomes for audiology students using virtual reality or traditional training methods”, PloS one, vol. 15, no. 12, 2020.

Clinical training requires students to gain experience with subjects exhibiting different medical conditions, which is challenging in a training lab. While peers often serve as test subjects for other students, this does not guarantee exposure to, and experience with, the full spectrum of medical conditions.

The American Speech-Language-Hearing Association recognizes the use of standardized patients and simulation technologies as alternatives to clinical education methods. See, ASHA, “2014 standards for the certificate of clinical competence in speech-language pathology”, Council for Clinical Certification in Audiology and Speech-Language Pathology of the American Speech-Language-Hearing Association., 2016, retrieved [6/1/2021] from asha.org/Certification/2014-Speech-Language-Pathology-Certification-Standards. Traditionally, standardized patients (SP) were individuals trained to portray a specific clinical case in a highly consistent and measurable manner. See, R. I. Zraick, “Review of the use of standardized patients in speech-language pathology clinical education”, International Journal of Therapy and Rehabilitation, vol. 19, no. 2, pp. 112-118, 2012. With the introduction of configurable manikins, different SP profiles may be loaded into a test manikin to present students with different scenarios. The National Council of State Boards of Nursing (NCSBN) also supports the use of quality simulations as a substitute for a percentage of clinical hours in undergraduate nursing programs. See, C. Nye, S. H. Campbell, S. H. Hebert, C. Short, and M. Thomas“, Simulation in advanced practice nursing programs: A North-American survey”, Clinical Simulation in Nursing, vol. 26, no. 3-10, 2019. Generally, little is known about the effectiveness of simulation in audiology education and training. See, W. A. Kaf, C. G. Masterson, N. Dion, S. L. Berg, and M. K. Abdelhakiem, M. K., “Optimizing otoscopy competency in audiology students through supplementary otoscopy training”, Journal of the American Academy of Audiology, vol. 24, no. 9, pp. 859-866, 2013, A. A. Alanazi, “The use of simulation in audiology education to improve students' professional competency”, ProQuest LLC, 2017, and A. Reed, A. Andre, S. Ananthakrishnan, and P. Korczak, “Effectiveness of simulation training on graduate audiology students' auditory brainstem response testing skills”, American Journal of Audiology, pp. 1-10, 2021.

Health care professionals need training in many areas, including communication with patients, diagnostic procedures, and how to work with appropriate diagnostic equipment. This is true for audiology education. Healthcare professionals need to be familiar with how to operate specialized diagnostic equipment and interpretation of results. However, gaining access to the diagnostic equipment can be difficult. There is a significant expense to equip and staff an instructional lab, which limits the hours the lab is accessible and the number of students that can receive training at any given time.

Hearing Screening tests are administered by those trained to do these tests, including speech audiologists, speech language pathologists, nurses, and teachers. See, ASHA, “Tests of the middle ear”, American Speech-Language-Hearing Association, 2021, retrieved 6/4/2021 from asha.org/public/hearing/tests-of-the-middle-ear/. A hearing screening is a quick, inexpensive, easily administered test that results in either a pass/refer result. Passing indicates the patient shows no signs of hearing loss. Not passing means the patient should be referred for a more thorough hearing assessment by a licensed audiologist.

The American Academy of Pediatrics (AAP) recommendations for preventative pediatric health care advocates regular hearing screening for all children from age 4 to 21 years of age, regardless of the presence or absence of risk factors for hearing loss. See, AAP, “Recommendations for preventive pediatric health care. Pediatrics”, Committee on Practice and Ambulatory Medicine. American Academy of Pediatrics, vol. 105, no. 3, pp. 645-646, 2000, retrieved on Jun. 4, 2021 from pediatrics.aappublications.org/content/105/3/645 and AAP, “2017 recommendations for preventive pediatric health care”, American Academy of Pediatrics, 2017, retrieved on Jun. 4, 2021 from pediatrics.aappublications.org/content/pediatrics/139/4/e20170254.full.pdf?embargo=true&download=true. This suggests that the 155,000 new nursing graduates each year need training in how to administer audiometric screening tests. In addition, there are over 4 million nurses in the U.S. and 20 million worldwide that would benefit from continuing education training in this area. California, for example, requires registered nurses to complete 30 hours of continuing education contact hours every 2 years to maintain their active license. See, CBRN, “Continuing education for license renewal, California Board of Registered Nursing, 2021, retrieved on Jun. 4, 2021 from rn.ca.gov/licensees/ce-renewal.shtml.

Getting the necessary audiology training can be challenging due to capacity limitations of the training lab, or the lack of a convenient facility for remote learners. To address these issues, the current disclosure devised a hearing screening simulator to help offset the training deficit. While simulation is an effective pedagogy for educating health professionals and is used extensively in undergraduate nursing education, it is not in widespread use in the field of audiology training. The inventors sought to design and develop a tool that could provide nursing students practice in performing hearing screening tests online, without the requirement to visit an “on-campus” hearing lab and interacting with live patients. This was particularly important during the COVID-19 pandemic where disease exposure risk was a critical issue. The capacity of the lab was limited in terms of both space and availability of certified trainers making scheduling difficult. Traveling to the physical training lab was also inconvenient for many of the remote students, who live all over North America.

A design science approach was adopted for this project. Initial design specifications were collected for the Hearing Screening Simulator. An iterative process involving the development of a prototype, review, and critique by the lab staff were employed which fed into the next development iteration. After several iterations, the final prototype was tested to assess if students felt the simulator was useful in learning how to administer hearing screening tests and determine students' confidence in their skills after using the simulator.

A national study found that 14.9% of children (6-19 years of age) have low- or high-frequency hearing loss in one or both ears. See, A. S. Niskar, S. M. Kieszak, A. Holmes, E. Esteban, C. Rubin, and D. J. Brody, “Prevalence of hearing loss among children 6 to 19 years of age: the Third National Health and Nutrition Examination Survey. Jama, vol. 279, no. 14, pp. 1071-1075, 1988. Early identification, diagnosis, and intervention are beneficial, improving the likelihood that children develop effective communication and language skills, and achieve successful learning outcomes. See, M. J. Guralnick, M. J., “Why early intervention works: A systems perspective”, Infants & Young Children, vol. 24, pp. 6-28, 2011. As such, the test must be carried out effectively. This is in part accomplished through adequate training of healthcare students and personnel on how to perform tests and diagnose patients. See, A. A. Alanazi, supra.

A. Hearing Screening Test Procedure

The American Speech-Language-Hearing Association's (ASHA) Guidelines for Audiologic Screening, see, American Speech-Language-Hearing Association, supra., specifies different procedures for doing screening for hearing impairment based on the age of the subject. There are guidelines for screening: newborns and infants age birth through 6 months; infants and toddlers age 7 months through 2 years; preschool children age 3 to 5 years, school-age children age 5 through 18 years; and adults 18 years or older. Of interest to the current disclosure are the guidelines for school-age children ages 3 to 5 ages, children ages 5 to 18 years, and adults 18 years and above. The younger subjects require additional training and different procedures. In part, the guidelines for subjects in the 5-to-18 year age group, specify testing should be done using 1000, 2000, and 4000 Hz tones at 20 dB HL, and that the test is repeated at least twice at each frequency to assure reliability. For adults, the testing should also be done using 1000, 2000, and 4000 Hz tones, but with a 25 dB HL setting.

ASHA's Guidelines for Audiologic Screening, see Id., also provides guidelines for screening the outer and middle ears. This procedure, which is the same for all age groups, includes an optional case history, visual examination (otoscope examination), and acoustic immittance testing (tympanogram).

Studies have shown simulation to be effective in audiology education and training. See, A. A. Alanazi, M. S. Mohamud, S. S. AlSuwailem, “The effect of simulation learning on audiology and speech-language pathology students' self-confidence related to early hearing detection and intervention: a randomized experiment”, Speech, Language and Hearing, pp. 1-14, 2020, R. Ozelie, P. Panfil, N. Swiderski, and E. Walz, “Hearing voices simulation: impact on occupational therapy students”, The Open Journal of Occupational Therapy, vol. 6 no. 4, 10, 2018, and E. Toader, “Clinical simulations for learning medical skills: a work-based approach to simulators”, Procedia-Social and Behavioral Sciences, vol. 197, pp. 2443-2448, 2015.

Simulation can help transfer theory to practice in an integrated teaching and learning model. See, B. M. Halm, M. T. Lee, A. A. Franke, “Improving toxicology knowledge in preclinical medical students using high-fidelity patient simulators”. Hawaii Medical Journal, vol. 70, no. 6, pp. 112-115, 2011. Simulators can present a full range of patient cases that represent real-world clinical diagnoses. These simulated cases present patients with symptoms that must be detected and diagnosed by the student. However, the use of simulation in audiology education is still in its infancy. See, ProQuest LLC, 2017, supra.

The inventors are not aware of any existing simulation tools that model the operation of commercial audiometry devices, or that provide the means for students to train on the use of such devices. CARL, sold by AHead Simulations, is a physiologically and acoustically realistic desktop manikin that enables clinicians and students to easily and safely train and develop their skills. “CARL is so realistic that you can see inside his ear, make measurements, fit hearing aids and tubes, and generate accurate readings that indicate how well the hearing aid is fitted.” Some of the applications that have used CARL include an introduction to audiometry and occupational hearing, serving as a standardized patient for audiometry and the fitting of hearing aid training, evaluating hearing aid streaming of music and speech, and the programming and verifying telecoil programs. However, CARL is suitable only for in-person training using commercial clinical test equipment since CARL simulates standardized patients, not clinical instruments. While CARL is a useful educational tool for audiologists who will go on to provide diagnostic evaluations and hearing aid programming, the technology is too complex and not suited for training providers who will go on to complete hearing screenings.

Prior work in audiology simulation education includes the use of an immersive training system that was found to provide audiology students with better learning outcomes and self-confidence than found with traditional training. See, D. Bakhos, et al. supra. Simulated training has been found to help with student's self-confidence in performing audiology testing. See, Id., and Speech, Language and Hearing, pp. 1-14, 2020, supra. The use of standardized patients for training on infant hearing screening and parental counseling simulation was found to improve clinical skills and increase confidence levels of audiology students. See, Id. In one study, significant improvements in clinical skill levels and higher confidence were observed when students completed the simulation task of preparing an adult mannequin for an auditory brainstem response (ABR) test. See, A. Reed, et al., supra. In another study, the incorporation of simulation workshops in Speech and Language Therapy curricula was found to have a positive quantitative and qualitative impact on students' perception of learning. See, M. M. Munoz-Montes, I. Villagrán-Gutiérrez, F. Pozo-Tapia, P. Tapia-Tapia, Y. Castro-Soares, and E. Fuentes-Lopez, “Speech and language therapy students' perception of learning through the incorporation of clinical simulation workshops: a mixed-methods study”, Revista CEFAC, vol. 23, 2021. The critical review and debriefing of students by qualified practitioners following simulated training is an important aspect of an experiential learning experience. See, R. M. Fanning, and D. M. Gaba, “The role of debriefing in simulation-based learning”, Simulation in Healthcare, vol. 2, no. 2, pp. 115-125, 2007.

The current disclosure follows a design science research (DSR) methodology consisting of (i) problem identification and motivation, (ii) definition of the objectives for a solution, (iii) design and development, (iv) demonstration, (v) evaluation, and (vi) communication. See, S. Gregor and A. R. Hevner, “Positioning and presenting design science research for maximum impact,” MIS Quarterly: Management Information Systems, vol. 37, no. 2. University of Minnesota, pp. 337-355, 2013, doi: 10.25300/MISQ/2013/37.2.01 and K. Peffers, T. Tuunanen, M. A. Rothenberger, and S. Chatterjee, “A Design Science Research Methodology for Information Systems Research,” J. Manag. Inf. Syst., vol. 24, no. 3, pp. 45-77, 2007, doi: 10.2753/MIS0742-1222240302. The current disclosure identified and translated design requirements for an audiology simulator into design components. The inventors then classified these functional components in the app into design principles and features for designing a healthcare simulation app for clinical education and training. The inventors conformed their meta-artifacts to mode 3C and 4B of design theorizing, i.e. seek to contribute to knowledge for solution design process and system to inform the design of the DSR project system and codify effective design principles and features, see, A. Drechsler, “Utilizing, Producing, and Contributing Design Knowledge in DSR Projects,” Open Access Victoria University of Wellington|Te Herenga Waka, January 2011. doi: 10.26686/WGTN.13088369.V1, that contribute to future DSR projects of similar context. The design requirements, artifact design and development, and evaluation are described below.

Hearing clinic personnel sought a tool to train students in an online Master's of Speech Language Pathology program on how acceleratorcentre.com/news/although-he-may-look-like-one-carl-is-no-dummy to administer hearing screening assessments. This involves performing three separate tests—otoscopy which takes a picture of the outer ear, tympanometry which measures the movement of the eardrum in response to a pressure change, and a pure-tone audiometer which measures hearing impairment. The clinic indicated that no such simulator existed, and the development would be useful, making it easier for students and practitioners to more easily train in performing these assessments. The goal of the current disclosure was to develop an effective simulator that could replicate as close as possible the experience of administering and interpreting a hearing screening test. To guide this disclosure, a conceptual model of the application was first developed.

Before an effective solution could be developed, it is necessary to understand the problem. Since the goal was to simulate an existing process, it was necessary to understand all the elements that play a role in that process and understand how they interact. To simulate a hearing screening test there are two actors, namely the technician administering the test, and the subject being tested. Also critical is the testing environment which includes the clinical equipment needed to perform the test. A less obvious element relates to systems that might address administrative requirements. These might be administrative or reporting functions that are done in the background. Developing an understanding of how actors interact is also critical. An effective approach is to develop separate Use Cases to detail how actors interact and what information is being transferred.

For the current project, there are five Use Cases, namely: authenticating and authorizing the technician, collecting patient history, following the proper protocol and performing the hearing screening, recording of technician notes during the examination, and assessing the accuracy of technician's examination process and subsequent results.

Design requirements were gathered in four iterations with a clinical assistant professor at the University of South Carolina Montgomery Speech-Language-Hearing clinic in the Arnold School of Public Health. A series of informal interviews were conducted to gather and clarify requirements at the outset of the project as well as during software development. An agile development model was used. Once an operational prototype was developed, the clinical instructor would evaluate it and provide feedback, which initiated the next round in the development. System specifications expanded as the project progressed. The initial object was to have a simulator that would give students exposure to the audiometer but automatically assess student performance. As the capabilities of the simulator increased, so too did the objectives of the project.

Meta-requirements fall into two categories—clinical requirements necessary for performing a hearing screening evaluation, and those included for operational reasons. The clinical requirements reflect the data that would be collected and presented using a traditional training session with commercial equipment. The operational requirements reflect additional features that exceed the clinical requirements and enable a web-based digital learning experience, such as calculate a student grade and therefore represent value-added aspects of the simulator. The Meta requirements included:

1) Clinical Requirements
Authenticate and authorize a user;
Graphical user interface (GUI) that has a similar look and feel to standard audiology testing hardware-based systems;
Ability to simulate three audiometry tests—video otoscopy, tympanometry, and audiometry;
Audiometer testing only needed for a specific subset of frequencies used for hearing screening;
Display output similar to that obtained with commercial devices, including otoscope images, tympanograms, and audiometer charts;
Include standardized patients representative of the range and scope of real-world clinical cases

2) Operational Requirements
Web-based platform;
Tracking and reporting of time spent on the simulation;
Ability to document and printout a record of the simulation session;
Provide automatic grading and feedback of a user's/student's performance;
Provide a full-featured audiometer that provides features beyond those needed for the hearing screening learning experience, but is present in commercial units;
Log all simulation testing to permit reporting of an individual student's testing history, and enable cross-sectional data analysis across multiple students' results.

Traditionally, software was developed for specific platforms, such as WINDOWS, LINUX, or MAC OS. Today, developers build Web-based applications that run on the Web, that are completely independent of the user's actual computer operating system. Web-based applications are a particular type of software that allows users to interact with a remote server through a web browser interface. They have seen a huge increase in popularity in recent years, replacing desktop applications and becoming a crucial instrument for small and large businesses around the world.

Web-based applications have a number of advantages over traditional desktop apps, most prominently their portability. With web-based apps, users don't have to install additional software, and developers do not have to write multiple versions of the same application for different operating systems. Various functions of the current disclosure are well suited to web-based applications. This include tracking performance records of students/trainees, managing lesson plans, monitoring progress on the HSS, and/or, not limited to, controlling work flow of students to alter the teaching pace, such as increasing or decreasing same, to cater to a particular student's training speed/learning rate. All of this can be achieved by writing a single web-based application that will be run on a server. The app can be written in various programming languages and make use of multiple technologies and frameworks. A web-based application will run on the user's computer's browser no matter what operating system is installed. This makes web-based apps one of the most universal cross-platform solutions available today.

Web-based software does not have to be installed and configured, so it is much easier to quickly increase the number of active users as opposed to desktop programs. Further, modern Web servers perform extremely well even when faced with thousands of simultaneous requests, so expanding the network of Web app users is often possible without any additional software configuration or modification, such as adding additional classes, remote learning facilities, etc.

A web-based application is any program that is accessed over a network connection using HTTP, rather than existing within a device's memory. Web-based applications often run inside a web browser. However, web-based applications also may be client-based, where a small part of the program is downloaded to a user's desktop, but processing is done over the internet on an external server.

A simulation objective was to get students familiar with the audiology equipment and comfortable administering hearing screening tests. Having a similar look and feel to commercial equipment was a high priority. To complete a hearing screening test, students need to be familiar with all three required testing modes (i.e., otoscope, tympanometer, and audiometer), so inclusion was important. Since the focus was on screening, and not a full hearing assessment, the system only had to simulate a limited set of test points. Being able to interpret test data and reach a correct result was also a key goal, and so it was important to present the results in an industry-standard format. The initial requirements did not include standard test subjects. This requirement was added in an early prototype review. The inventors started with a single subject, which was eventually expanded to ten.

Of the operational requirements, porting the simulation was a high priority. The disclosure may function through Windows® applications and is stable and performing as expected. With this goal of moving the simulator to the web, the inclusion of extra Metadata that reports time spent and the number of tests completed will help give training supervisors confidence that the student has completed the training. Being able to print out the session record lets the students document their performance. Automatic grading provides the learner immediate feedback and reinforces the learning process. Having a full-featured simulator allows the curious learner to explore on their own, and also allows the simulator to be used for training other than for just hearing screening (i.e., completing more comprehensive audiology testing). Also having the ability to assess results across multiple student technicians may help identify weaknesses and strengths in the classroom training.

The Hearing Screening Simulator (HSS) was developed as a Windows form application using Visual Basic as the development language. Visual Basic was used because it supported a graphical user interface, integrated charting libraries needed for the audiology graphs, and the ease with which it could be ported to a web-based platform. The design of the simulator unit was based on the look of existing audiology testing units in the lab. This was done to acquaint students with the layout and operation of these devices and to provide a more realistic simulation experience.

The HSS is designed to simulate three clinical tests used in hearing screening and audiological evaluations. An otoscope exam helps to assess the condition of the external auditory canal and tympanic membrane. This is a visual process. A doctor may use a traditional otoscope to look into the patient's ears, or, as in our simulation, use a camera to capture an image. Tympanometry tests how well the subject's eardrum moves, as well as determine the volume of the ear canal and pressure of the middle ear space. The audiologist will put a small probe, which looks like an earphone, into each ear. A small device attached to the probe will push air into the subject's ear, and the device measures the resulting response. This creates a graph, which is displayed on the test unit. See, ASHA “Tests of the middle ear,” supra. The audiometer is used to determine the quietest sound the subject can hear at different pitches or frequencies. A tone is presented and the subject indicates if they heard it by raising their hand. In a screening, however, tones are presented at a fixed level considered “normal hearing” (20 dBHL), rather than determining the softest level the subject can hear.

All student interaction with the simulator is stored in a SQL database. This also handles user authentication and authorization. The simulator user interface consisting of three panels is shown in FIG. 1. The left panel presents the simulated audiometer device. Similar to commercial audiometers designs, the upper portion of the unit contains a graphic display that shows either the tympanogram or audiology chart depending on the testing mode selected. The lower portion of the unit provides the various controls used to perform the testing. These controls and their purpose are described below. The Center section provides information about the subject, and command buttons to display and print a larger version of the audiogram chart, and to display the test debriefing report, which summarizes the student performance on the simulation. The right panel is for Technician Notes. It allows the student to record their findings and include observation as is typical in a traditional audiological screening.

The user interface is designed with interactive objects, see FIG. 1. As buttons are pressed the simulator provides sensory feedback to let the user know the action was registered. For example, when the user selects one of the three buttons in the top left, it selects the testing mode and toggles the button color (blue indicates that option is selected). This is accomplished by loading either the selected or unselected image into the object when a button is pressed. The audiology chart was created using the native charting capability of Visual Basic.

The simulator's three testing modes are ‘Oto’ to display an otoscope image, ‘Tym’ to display the tympanogram of the target ear, or ‘Aud’ to perform an audiological test. The bank of buttons on the right control the test conditions for the audiometer. This includes frequency in Hz, intensity level in dBHL, sample type (being either pure tone, pulsed, or frequency modulated), and controls to specify which ear is to be tested (right, left, or both (stereo). The large dial on the bottom left provides an alternate way of controlling the intensity level presented to the subject. The intensity level is graphically displayed in the progress bar above the dial.

Modeled on the design and operation of commercial devices, the curved button is referred to as the ‘Present’ button, which serves one of two functions based on the testing mode. On commercial devices a tympanogram test begins automatically when a seal is obtained on the ear canal. Since the simulator did not model acquiring this seal, there needed to be a way to initiate the test. The present button was used to trigger the test and displays the resulting tympanogram chart. There is no operator or subject response needed for this test—the test is fast and automatic. The HSS displaying a stored image file representing the subject's tympanogram chart after the student presses the present button. it is meant to simulate a tympanogram test. When the user presses the curved Present button in the ‘Aud’ (audiometer mode) the test tone is presented to the subject. To provide visual confirmation that the tone was played, an indicator light is displayed next to the avatar. Note, this is meant as an indicator to the technician, and no such indicator would be visible to the subject in real audiological tests.

The subject avatar raises its hand if the simulation ‘hears’ the presented tone, see FIG. 4. If the avatar raises their hand, the technician presses the Response button indicating the subject responded to the tone. HSS records that the subject responded to the signal at the specific frequency and decibel level. If there is no response, the tone can be replayed, or the decibel level increased until the subject does give a response. The test process frequency can then be indexed to the next test point and the process repeated. This process records the subject's response on the audiogram at the top of the simulator. Data for the right, left and both ears (stereo) are included on the same chart. The technician has the option to enlarge and print the diagram using the controls in the center panel.

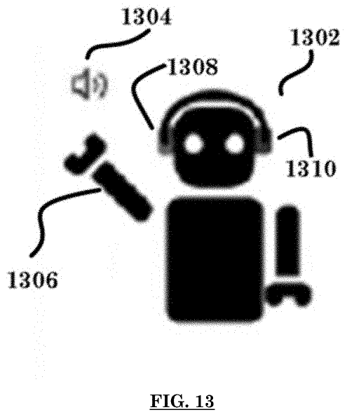

In the audiogram mode, when a tone is presented to the subject, a representative tone is also played for the technician (i.e., the student) and an indicator light illuminated, see FIG. 13. FIG. 13 shows avatar 1302 and a sound indicator 1304 showing the user the subject was presented with a sound and heard same as shown by the avatar raising its right arm 1306. The frequency and intensity of the tone played through the computer's speakers are also modulated to simulate what the subject is hearing. To model the operation of actual audiology devices, the simulator can perform testing using constant tones, pulsed tones, or frequency modulated samples. Testing can be done on the right ear 1308 or left ear 1310, or the stereo setting can be used to present the tones in both ears simultaneously. When the simulator is in the ‘Tym’ (tympanogram) mode, the tympanogram image of the target ear is displayed in the graphic display at the top of the unit. There are controls to change the testing frequency between nine specific values between 500 Hz and 8000 Hz. Some controls allow the operator to modulate the sound intensity level from 10 dBHL to 70 dBHL.

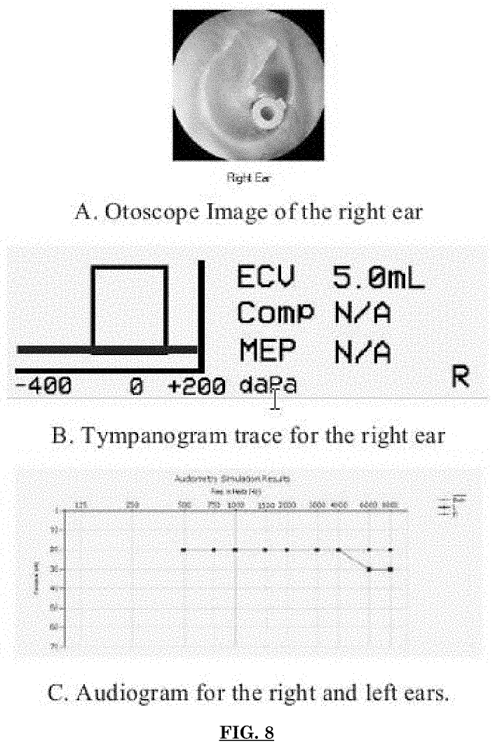

A sample otoscope image, tympanogram, and audiogram chart are shown in FIG. 8 at (a), (b) and (c), respectively. Note, the otoscope images and tympanograms are specific to the subject's ear being tested. Those images are part of the subject profile that is loaded when the subject is selected. In contrast, the audiogram is dynamically created based on the technician's recording of the subject's responses. The chart shows data from both ears on the single graph. Note, the audiogram shows subject responses at more frequencies and intensity levels than used for screening tests.

The HSS simulator may be equipped with a number of patients, such as equipped with 10 (or more or fewer) standard patients, although the set of standard patients could also be expanded to enhance the catalog of clinical cases covered by the simulator. Each patient has a short backstory which is presented to the student when the subject is selected. This provides the age and gender of the subject, and a brief background comment related to any hearing concerns. The ten subjects were created with varying audiologic conditions and pathologies, with audiometry, tympanometry, and otoscopy results consistent with their condition. One child and adult standard patient with normal results were included as well. To help mask the identity of these standard patients and give the illusion of having a larger subject base, the profile information is randomized each time the program is run. The genders are changed, age shifted slightly, the name of the subject being selected from a large list of diverse names, and the backstory adjusted to match the new subject profile. This not only prevents someone from circumventing the training by utilizing results from earlier tests, but it also enables repetitive testing on the same standardized subject without it being evident it is the same subject.

As the student goes through the simulation, they are asked to record their observations and patient results in the Technician Notes panel. There are note sections for each of the three tests (otoscope, tympanogram, and audiometer). The student is asked to provide a result for each ear and to add comments and observations related to that test. Having the student enter this information into the simulator allows the simulator to assess their performance. The system knows which result is correct based on the standard subject being tested. Taking into consideration the three individual tests, the student must also make a recommendation regarding the patient's subsequent follow-up, and also has the opportunity to add a related comment. This final recommendation can also be automatically graded by the simulator and provide appropriate feedback as needed. The patient cases, associated correct result, and correct recommendations were defined by the project clinical team and implemented in the application database.

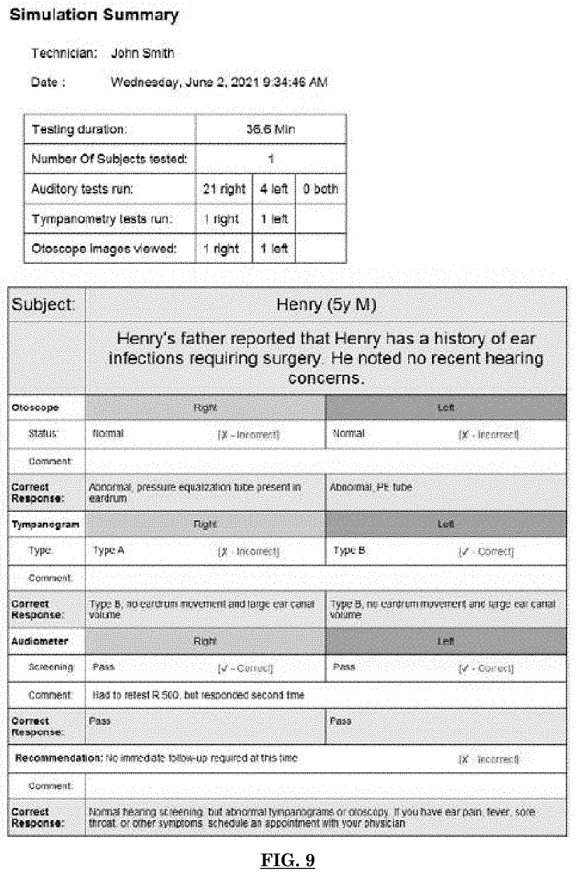

Once the student completes the testing and enters their session notes, the data is saved to a database and a session report is generated, see FIG. 9, a simulator summary for the subject being tested. This report serves as a debriefing for the simulated training. It provides Metadata related to the testing, including the technician's name (i.e., the student's name), the subject's name, the duration of testing, which tests were run during the evaluation, as well as the total time spent using the hearing screening application. This session report provides information to the training supervisor to show what steps the student went through during the session. If the student took shortcuts during the session, this data would make that evident. The report also shows the student's results along with the correct results coupled with an explanation justifying the result. This gives the student immediate feedback on their performance. These reports can be printed out or saved as a pdf.

Thirty-three first year speech-language pathology graduate students enrolled in an introduction to audiology class participated to help refine this disclosure. All participants received the same standardized lecture covering concepts surrounding hearing loss and how to perform hearing screening assessments. Students were randomly assigned to the control (n=16, female=16) or test (n=17, female=17) groups. The control group received brief in-person training to explain how to operate the equipment, and then were evaluated conducting their live, in-person audiology test. The test group received the same in-person equipment introduction and used the simulation tool before being evaluated doing their live audiology test. Students in each group were divided into two-member teams, with one group of three in the test group. Each student team rotated between roles, acting as a test subject (i.e., patient) and acting as the test evaluator. Approximately half of the students in each group had previous hands-on experience with a hearing screening audiometer. Students were sorted into an approximately equal number of pairings of “no previous experience,” “one partner with previous experience”, and “both partners with previous experience”.

Participants in the test group received supplementary training with the simulation tool. They were introduced to the simulator and were allowed to work with it for about 15 minutes, working through approximately five cases with their partner. A 25 item questionnaire was administered to students to assess their confidence in their skills both prior to and after performing the live hearing screening test. The questionnaire was created by the audiology staff to gauge students' knowledge and confidence in administering and interpreting hearing screening tests. A five-point Likert scale was used. Response options and subsequent coding values were Strongly Disagree (1), Disagree (2), Neither Disagree or Agree (3), Agree (4), and Strongly Agree (5). A set of seven to nine questions were asked related to each of the assessment modes (otoscope, tympanogram, and audiometer). The test group was also asked four additional free response questions in the same questionnaire to assess perceptions about the simulation tool. A total of 16 surveys were completed by each group. One survey was not usable and was thus removed from the analysis.

A live assessment of student clinical technique was completed using commercial audiometry units using peers as test subjects. Supervising certified instructors observed the tests and graded the student's performance and technique.

It was expected that using the simulation training tool together with in-person training would be perceived by students to be at least as effective, and possibly an improvement, over in-person only training using commercial audiology equipment. Though some indication of improvement was expected, statistically significant differences between groups were not expected for most questionnaire items between the test and control groups. All statistical analyses were conducted using a standard, commercially available statistical software tool (NCSS 2019 Statistical Software). Thematic analysis of qualitative responses was conducted to produce categorical findings and related discussion.

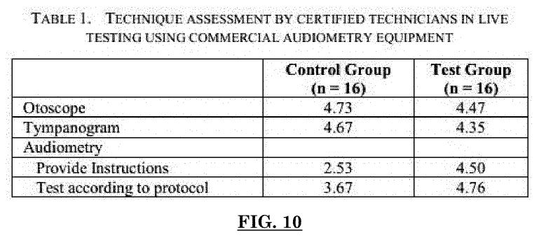

Supervising certified instructors observed the student testing and graded the student's performance and technique. All students in both the control and test group performed the three tests satisfactorily (i.e., otoscopy, tympanogram, hearing screening). Some students, primarily in the control group, did not follow the prescribed protocol exactly, as reflected in the technique scores shown in Table 1, see FIG. 10. Students performed similarly across test and control groups except in the case of providing instructions and testing according to protocol.

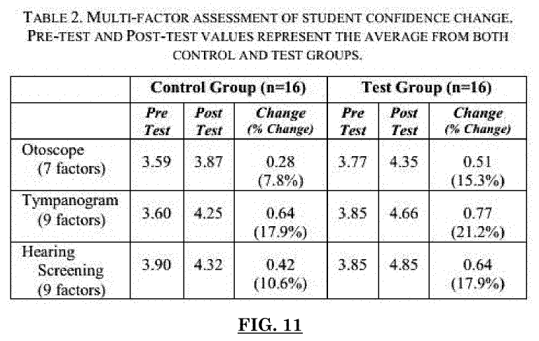

The test group resulted in a higher post-test mean score and a higher percent change than the control group for all three test component groupings: Otoscope, Tympanogram, and Hearing Screening (see Table 2, FIG. 11). To test for statistical significance between groups, a non-parametric analysis was conducted using the Kruskal-Wallis one-way ANOVA to test differences between the change in test and control group pre-test vs post-test survey responses (post-test mean score minus pre-test mean score). The test was corrected for tied ranks.

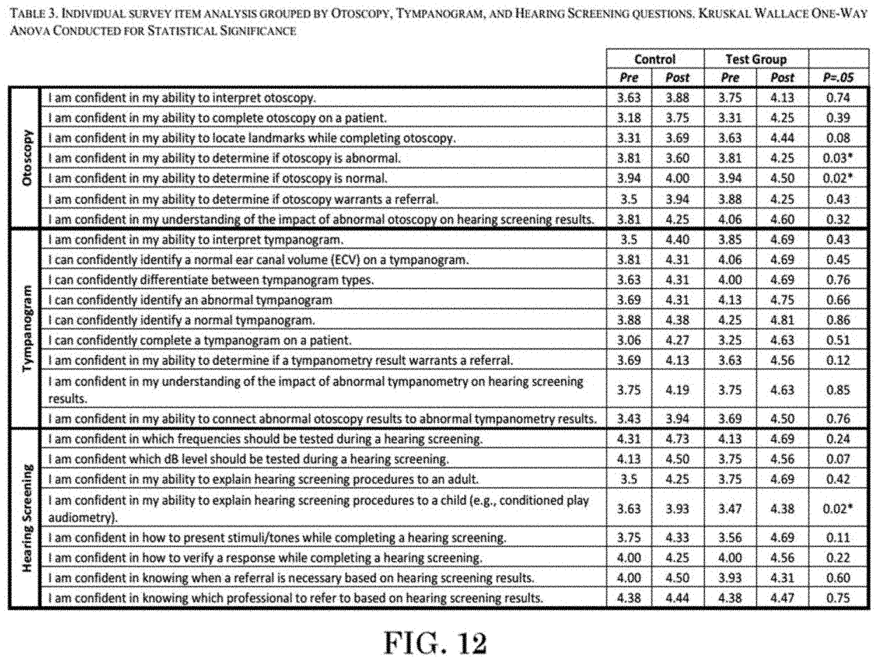

As shown in Table 3, see FIG. 12, three question responses showed statistically significant differences between groups, including: “I am confident in my ability to explain hearing screening procedures to a child (Chi square=5.27, p=0.02, df=1);” “I am confident in my ability to determine if otoscopy is normal” (Chi square=5.14, p=0.02, df=1); and “I am confident in my ability to determine if otoscopy is abnormal” (Chi square=4.86, p=0.03, df=1). Results were not significant across 22 survey questions at the p=0.05 significance level and thus showed no statistically significant difference attributable to the use of the simulator.

The test group was asked four free response questions regarding their impression of the simulator tool. The first asked “what aspects of the simulation did you like?” The responses were grouped into common themes, namely:
Provides hands-on experience, helped me become more familiar with an audiometer & increased my confidence (5 responses);
Ease of use (4 responses);
Practice with multiple virtual patients (4 responses);
Provided useful feedback on the test debriefing report (1 response);
Thought it was helpful (1 response).

The responses indicate that the students found the hands-on experience provided by the simulator provided an easy to use and useful learning experience with the audiometer that increased the students' confidence in their ability to perform hearing screening testing.

The second free response question asked “what aspects of the simulator did you not like?” This also elicited five thematic responses, namely:
Did not show how to do otoscopic or tympanometry exam (4 responses);
Having better instructions would be helpful (4 responses);
Nothing, this is a useful simulation (2 responses);
It was repetitive (1 response); and
Did not look like the audiogram machine (1 response).

The simulator was designed to display an image or chart for a virtual subject and not how to perform an otoscopic or tympanometry exam. Students wanted more explanation as to how to perform these exams. Others felt the instructions, in general, could be improved, possibly incorporating pictures or a video. One comment expressed a concern that the design of the simulator did not look like the audiogram machine. Given the simulator was modeled to look similar to the GSI 39 Combination Audiometer and Tympanometry device, it is assumed the student was familiar with a different model.

The third question asked “do you have any recommendations to improve the simulator?” Response themes were:
The simulator did not show how to perform otoscope and tympanometry. Clearer instructions are needed in general (5 responses)
Nothing, no improvement needed. (3 responses)
Model the technician giving required directions to the subject (1 response)
Have the design of the simulator match that of the audiometer (1 response)

The most frequent response was that the simulator should improve training on how to perform otoscopy and tympanometry testing. It was also suggested that the simulator model the instructions provided by the testing technician to the subject. Future software iterations may consider these changes.

The fourth question asked the student “would you like to share anything else?” Responses included:
“A great tool to add to any audiology class” (1 response)
“Really liked that sample participants showed inconsistencies in hearing thresholds. This realistic touch prepares one to be not thrown off when performing a live, in-person test.” (1 response)
“The simulation was easy to use” (1 response)
“The virtual simulation was very helpful” (1 response)
“I loved the activity!” (1 response)

Responses to the final question noted positivity towards the simulation tool indicating its potential benefit for providing a range of realistic learning experiences. It does suggest however that some feature of the simulator need to be discussed further in the system documentation. The final three comments reinforced that the simulator was easy to use, and perceived as a useful addition to the class.

The HSS disclosed herein is a novel solution that may be useful in training future Speech-Language Pathologists, student nurses, and other professionals in the performance of hearing screening testing. This is the only known solution that permits learners to perform a hearing screening assessment on simulated patients, and receive constructive feedback on their performance. It addresses a need for remote training that cannot be provided using commercial audiology equipment, and thus represents an improvement, see, S Gregor et al, supra, on the state-of-the-art. It provides a heretofore unavailable avenue for experiential learning for online and remote learners. The design science research framework provided in this study may be useful for developing simulation training, generally, and audiology simulation training specifically. The software simulation tool provides a design artifact that was tested and evaluated across 32 participants and thus provides a complete DSR cycle inclusive of artifact evaluation.

This disclosure provides a comprehensive technical investigation and evaluation of the requirements for the simulator focused on the user interface design, and user experience influencing the system design artifact instantiation of an HSS. In the evaluation, we surveyed users to assess usability, and efficacy as a teaching tool. While the mean scores for the test group were largely higher than the control group and demonstrated larger percent change from pre-test to post-test, there were few statistically significant differences between groups. This was not surprising considering the very early phase of research in this disclosure and the known difficulties measuring educational interventions especially with small sample sizes. Taken together, the quantitative and qualitative results from this disclosure show significant promise for audiology simulator tools for enhancing audiology education. The results of this disclosure indicate the working prototype of the simulator may provide a strong supplement to in-person training using commercially available equipment. Participants noted the simulator was intuitive and useful for learning the skills needed to perform hearing screening tests while also indicating areas for improvement.

While the number of participants is low (n=33), yet this is appropriate for an initial pilot test. Future studies should include a broader range of participants. This disclosure represents the first test of the simulation software. Future iterations of the software with prescribed changes may demonstrate different results. A common issue for the control group was failure to communicate instructions to the subjects as prescribed in the protocol. This was not as prevalent in the test group which may indicate the added simulator practice impacted the student's performance even though it did not explicitly indicate or model this behavior.

Various modifications and variations of the described methods, systems, and devices of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. An audiology simulator comprising:
an otoscopy component to provide a visual representation of at least one ear;
a tympanometry measuring component to simulate measuring eardrum movement in the at least one ear; and
an audiometer component to simulate measuring hearing in the at least one ear; and
wherein the otoscopy component, the tympanometry measuring component, and the audiometer component replicate administering and interpreting a hearing test given to a subject.

2. The audiology simulator of claim 1, wherein the audiology simulator provides at least one result of an operator's performance using the audiology simulator.

3. The audiology simulator of claim 1, wherein the audiology simulator enables cross-sectional data analysis across multiple users to compare different users' results using the audiology simulator.

4. The audiology simulator of claim 1, further comprising a graphical interface.

5. The audiology simulator of claim 1, wherein the graphical interface provides at least one display output that includes at least one otoscope image, at least one tympanogram, and at least one audiometer chart.

6. The audiology simulator of claim 1, wherein the audiology simulator is web-based.

7. The audiology simulator of claim 1, further comprising a subject information section providing information about the subject.

8. The audiology simulator of claim 1, wherein the audiology simulator provides sensory input to a user via interactive objects to confirm an action performed by the audiology simulator.

9. The audiology simulator of claim 1, further comprising an avatar representing the subject being tested by the audiology simulator wherein the avatar provides visual cues to a user to show the avatar receiving sensory input from the audiology simulator.

10. An auditory training simulation method comprising:
providing an audiology simulator comprising:
an otoscopy component to provide a visual representation of at least one ear;
a tympanometry measuring component to simulate measuring eardrum movement in the at least one ear; and
an audiometer component to simulate measuring hearing in the at least one ear; and
replicating administering and interpreting a hearing test given to a subject via the otoscopy component, the tympanometry measuring component, and the audiometer component.

11. The auditory training simulation method of claim 10, further comprising providing via the audiology simulator at least one result of an operator's performance using the audiology simulator.

12. The auditory training simulation method of claim 10, further comprising enabling, via the audiology simulator, cross-sectional data analysis across multiple users to compare different users' results using the audiology simulator.

13. The auditory training simulation method of claim 10, further comprising providing a graphical interface to interact with a user.

14. The auditory training simulation method of claim 13, further comprising providing via the graphical interface at least one display output that includes at least one otoscope image, at least one tympanogram, and at least one audiometer chart.

15. The auditory training simulation method of claim 10, further comprising providing the audiology simulator as a web-based platform.

16. The auditory training simulation method of claim 10, further comprising providing a subject information section that provides information about the subject.

17. The auditory training simulation method of claim 10, further comprising providing sensory input, via the audiology simulator, to a user via interactive objects to confirm an action performed by the audiology simulator.

18. The auditory training simulation method of claim 10, further comprising displaying, via the audiology simulator, an avatar representing the subject being tested by the audiology simulator wherein the avatar provides visual cues to a user to show the avatar receiving sensory input from the audiology simulator.
